(19) United States
(12) Patent Application Publication
(10) Pub. No.: US 2024/0285438 A1
(43) Pub. Date: Aug. 29, 2024

(54) SLEEP MASK

(71) Applicant: NETAWORK LIMITED, Sha Tin (HK)

(72) Inventor: Hok Leung CHOW, Sha Tin (HK)

(21) Appl. No.: 18/585,705

(22) Filed: Feb. 23, 2024

(30) Foreign Application Priority Data
Feb. 23, 2023 (HK) 32023068864.2

(51) Int. Cl. A61F 9/04

(52) U.S. Cl. CPC A61F 9/04

(57) ABSTRACT

A sleep mask detects the volume fed into the ear of the audio player through the sensor. If an unbalanced volume signal is detected in the two ears, it will be detected by the control module and the audio will be adjusted to output to the audio player and then let the volume balance while the audio enters two ears, thereby reduce the unbalanced volume problem caused by the volume change in one ear which may cause by the pressure on one ear when the user adjusts the posture, and reduce or eliminate ear discomfort and impact on sleep.

### FIELD OF THE INVENTION

The present invention involves a sleep mask, especially a sleep mask that helps to relax and fall asleep.

### BACKGROUND OF THE INVENTION

In modern, people often suffer from sleep disorder symptoms such as insomnia due to high pressure in work and life. There are many ways to improve insomnia, such as through drugs or physical methods. Although taking drugs is directly effective, long-term or excessive use of drugs can cause dependence and even side effects. The physical method is to help the human body into a relaxed state before going to sleep and isolate the influence of the outside world to improve insomnia. Of course, there are many possible physical methods, and sleep masks are one of the most common products currently on the market to help sleep.

By wearing sleep mask, on the one hand, it can reduce the external light stimulation to human eyes and promote the pineal gland to secrete melatonin. Melatonin's well-known function is mainly to improve sleep quality and shorten the time of awaking and falling asleep, significantly reduce the number of awakenings during sleep, etc. On the other hand, the sleep mask can also be equipped up with an audio playing device, which plays softer and relaxing sounds or music to relax the body and effectively improve sleep. However, when user fall asleep, he turn his body sideway or sleep on his side, which will cause part of the audio playing device to be squeezed, and the volume of the squeezed part is changed, thereby affecting the volume imbalance between the two ears. Such volume imbalance will produce an uncomfortable feeling for both ears, which may affect falling asleep or even wake the user up due to sudden volume changes.

### SUMMARY OF THE PRESENT INVENTION

The present invention provides a sleep mask, according to the sleeping posture or state to analysis the unbalanced volume of the two ears, then promptly adjust the unbalanced volume signal to restore balance to avoid affecting the user's sleeping status.

The present invention provides following technical resolutions:

A sleeping mask comprises a mask body for covering the eyes;
fixing pieces for fixing the mask body on the head;
the mask body at least extends from the eyes to the ears; a control circuit is provided inside the mask body and includes at least an audio control module;
the audio control module includes an audio player, the audio player is set at the positions corresponding to the ears;
the audio control module is also connected to at least a sensor for monitoring whether the volume fed into the ears by different audio players is balanced, the sensor transmits the unbalanced volume signal fed into the ear to the audio control module, the audio control module processes the unbalanced volume signal and the audio players are adjusted to make the ears receive a balanced volume signal.

Further, the sensor is a gyroscope and is connected to the I/O port of the audio control module.

Further, the sensor is a pressure sensor, which is disposed at a position of the mask body corresponding to the ears, and is connected to the I/O port of the audio control module.

Further, the sensor is a microphone for picking up feedback arranged at a position corresponding of the mask body corresponding to the ears, and the microphone is connected to the I/O port of the audio control module.

Further, a lighting device for guidance and/or waking up is provided on the inner side of the mask body at a position corresponding to the eyes.

Further, the mask body is composed of a front mask body and a rear mask body; the front mask body and the rear mask body are made of a sponge pad; and fixing slots for installing the control circuit and the audio player are formed in the front mask body and/or the rear mask body.

Further, the slot for installing the control circuit extends to the edge of the mask body and forms an opening; the control circuit is inserted into the mask body from the opening and fixed.

Further, the outer sides of the front mask body and the rear mask body are respectively covered with fabric layers and the edges of the fabric layers cover the edges of the sponge pad of the front mask body and the rear mask body, and then glue up the front mask body and the rear mask body.

Further, a window for installing the lighting device is provided on the inner side of the mask body facing to the eyes.

Further, eye pads are provided on the inner side of the mask body at a position corresponding to the eyes; the eye pads are annular gaskets arranged around the outline of the eyes; the eye pads are detachably installed on the inner side of the mask body.

Further, the mask body is provided with mounting grooves at a position where the eye pads are installed, and the eye pads are detachably fixed in the mounting groove through a magnetic attraction structure.

Further, the fixing pieces include headbands extending from both sides of the mask body, and the headbands are connected by Velcro tape; the Velcro tape comprises of a hook sticking tape and a loop sticking tape, and a barrier layer is set on the hook sticking tape and the is configured to be compressible, when under the non-compressed state, the barrier layer prevents the hook sticking tape from hooking up with the loop sticking tape; when under the compressed state, the hook sticking tape can hook up with the loop sticking tape.

Further, the height of the barrier layer is equal to or higher than the height of the hooks in the hook sticking tape.

Further, the barrier layer is distributed in the hook sticking tape according to a set pattern.

Compared with the existing technology, the present invention has the following advantages: the sensor is used to detect the volume fed into the ears by the player. If an unbalanced volume signal is detected between the two ears, the signal will be feed backed to the audio control module and the audio signal will be adjusted before being output to the audio player so that the volume of both ears is rebalanced, thereby reducing the unbalanced volume problem caused by the volume change in one ear due to pressure when the user adjusts the posture, reducing or eliminating ear discomfort the effect on sleep.

### BRIEF DESCRIPTION OF THE DRAWINGS

In order to illustrate the technical solutions in the embodiments of the present invention, below are explanations of the invention combining with drawings.

FIG. 1 is the perspective view of the present invention.

FIG. 2 is an exploded view.

FIG. 3 is another exploded view from other angle;

FIG. 4 is the schematic view of the Velcro tape.

### DESCRIPTION OF THE EMBODIMENTS

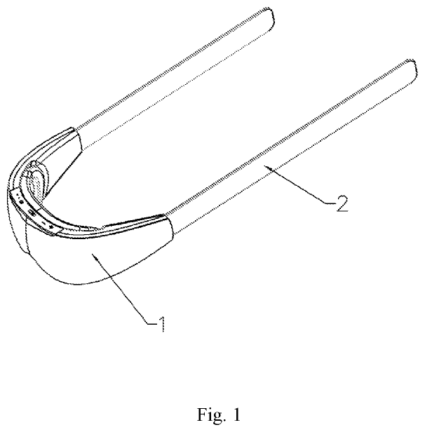

Refer to FIG. 1, the sleep mask, a mask body 1 for covering the eyes and fixing pieces 2 for fixing the mask body 1 on the head. The mask body 1 at least extends from the eyes to the ears. A control circuit 3 is provided inside the mask body 1 and includes at least an audio control module. The audio control module includes an audio player 4, the audio player 4 is set at the positions corresponding to the ears. The audio control module is also connected to at least a sensor for monitoring whether the volume fed into the ears by different audio players 4 is balanced, the sensor transmits the unbalanced volume signal fed into the ear to the audio control module, the audio control module processes the unbalanced volume signal and the audio players 4 are adjusted to make the ears receive a balanced volume signal. The audio player 4 mentioned above is generally a speaker. Of course, in order to reduce the size and weight of the entire sleep mask, a smaller and lighter speaker needs to be used. The sensor is used to detect or estimate the volume fed into the ears by the player. If an unbalanced volume signal is detected between the two ears, the signal will be detected by the audio control module and the audio signal will be adjusted before being output to the audio player 4 to rebalance the volume entering both ears, thereby reducing the unbalanced volume problem caused by the volume change in one ear due to pressure when the user adjusts the posture, reducing or eliminating ear discomfort the effect on sleep.

In order to identify whether there is an unbalanced volume signal, the sensor can detect it in a variety of ways. Different detection methods will use different type sensors and analysis methods. The various sensors are described in detail below.

One solution is that the sensor is a gyroscope, so a suitable gyroscope integrated chip can be selected according to actual needs. Generally, a gyroscope with two axes can meet the needs. Connect the corresponding I/O port of the gyroscope to the main control chip in the control circuit. When a person's head turns sideway, the gyroscope can promptly detect the position and angle of the rotation, etc., and use the position and angle data to determine whether there is pressure on the ear, which will cause the problem of the audio playing volume imbalance on both ears. Generally, the volume of the ear under pressure will be increased. If the volume of the two ears is unbalanced, the control circuit 3 outputs a control signal to the audio control module to reduce the audio playing volume of the pressured ear so that the volume of the two ears can be balanced again. Since the gyro sensor does not directly detect volume changes in the ear, designer can estimate the severity of unbalanced volume according to his experience by the data changes in the position and angle of the gyroscope, and then adjust to an appropriate volume value. The gyroscope chip can be directly arranged in the control circuit 3, thus simplifying the overall design of the system.

One more solution is that the sensor is a pressure sensor which is set at the mask body 1 of the position corresponding to the ear, and then connects the I/O port of each pressure sensor to the control module. As mentioned above, the most common situation is that the volume of the ear will increase when the ear is under pressure. Then by setting a pressure sensor on the ear, it can directly detect the pressure of the ear. The main control chip of the control module can detect whether it is under pressure and the severity of pressure based on the pressure change signal, etc., and then reduce the volume of the pressured ear to achieve the volume balance of both ears.

One more solution is that the sensor is a microphone for picking up feedback arranged at a position of the mask body corresponding to the ear, which directly receives the audio entering the ear through the microphone. By comparing the strength of the signals received by the microphones in both ears, it can be determined whether there is an unbalanced volume happened. Then the main control chip of the control module can adjust the volume of the ears respectively according to the volume signals received by the microphones to achieve the volume balance between the two ears. The advantage of this solution can detect the unbalanced volume caused by pressure on the ear, and can also detect the increase in volume caused by sound reflections by objects outside the ear, so it can be applied to more scenarios.

Wearing the sleep mask can prevent external light from entering the eyes, which is good for sleeping. However, because the external light is completely blocked, the eyes cannot sense changes outside, such as dawn, etc., which can easily lead to missing the time to get up. Although you can use other devices such as alarm clocks, mobile phones, watches, etc. to wake you up, it would be more ideal if you could use a sleep mask to achieve similar functions. Since the sleep mask is equipped with an audio player, as the wake-up time is set, the alarm clock function can be realized by playing gentle audios. At the same time, it can simulate changes of light by setting a lighting device on the inner side of the mask body 1 at a position corresponding to the eyes 6, by combining the audio player together with the lighting device 6, a better awakening effect can be achieved, and the eyes can easily adapt to the light changes in the outer environment while the sleep mask is removed. In addition, user who is in a state of mental stress and need to relax his tension to fall asleep. Although the sleep mask is already equipped with an audio player which can help relieve tension by playing relaxing and soft sounds or music, it can combine with breath training to improve the effect. So, the above-mentioned lighting device 6 can also serve as an instruction to guide breath training. Through a certain rhythm of light up and dark out changes, the user follows the light to adjust breathing, which can further relieve his nervousness and achieve falling asleep more smoothly. Moreover, the lighting device 6 is controlled by the control circuit 3, and can achieve changes in light and dark, color, gradient, etc., and can be freely adjusted according to different scenarios. Therefore, the lighting device 6 is not limited to sleep or wake-up functions. In order to facilitate the installation of the lighting device 6, a window for installing the lighting device 6 provided on the inner side of the mask body 1 facing to the eyes, see FIG. 3.

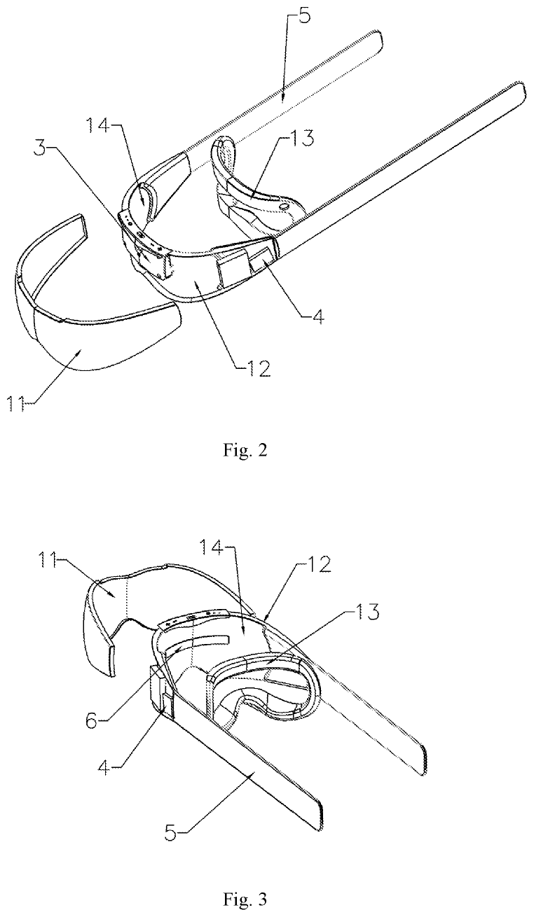

In order to facilitate the above-mentioned electronic devices, such as the control circuit 3, audio player 4, sensor and lighting device 6, they can be conveniently installed in the mask body 1, taking into account the convenience of assembly and the aesthetics of the arrangement, the inventor has designed a more reasonable structure of the mask body 1. The mask body 1 is composed of a front mask body 11 and a rear mask body 12, so that some of the above-mentioned electronic devices can be installed between the front mask body 11 and the rear mask body 12. And the combination sealing can be achieved in many ways, such as gluing, sewing or heat sealing, etc., refer to FIG. 2 and FIG. 3. Considering the comfort wearing of the user, the front mask body 11 and the rear mask body 12 are at least formed by sponge pads. In addition, in order to facilitate the assembly worker to quickly and accurately install the electronic device to the appropriate position, the front mask body 11 and/or the rear mask body 12 are molded for placing the control circuit 3 and the audio player 4. The fixing slots will be designed according to the overall contour and size of different electronic devices, so that the placed electronic devices can better fit the fixing slots for better fixation, and on the other hand for preventing protrusion of the electronic device from the mask body 1, causing wearing discomfort and affecting the overall aesthetics.

Because some electronic devices, such as the control circuit 3, are difficult to be assembled into the mask body 1 at one time, and some buttons and the charging interface of the control circuit 3 need to be exposed on the surface of the mask body 1 for user operation. Therefore, an opening is provided at the edge of the fixing slot of the control circuit 3 of the mask body 1. After the assembly of the front mark body 11 and the rear mask body 12 of the mask body 1 is completed, the control circuit 3 can be installed into the corresponding fixing slot by the opening, then connect other electronic devices to the control circuit 3 through the interface, and finally fix the control circuit 3 in the fixing slots to complete all installation procedures.

In order to further improve the overall aesthetics of the sleep mask, the outer sides of the front mask body 11 and the rear mask body 12 are respectively covered with fabric layers. User can choose different fabric layers according to his preference to obtain his favorite appearance of the sleep mask. After covering the edges of the sponge pads of the front mask body 11 and the rear mask body 12 with the edge of the fabric layer, the front mask body 11 and the rear mask body 12 are pasted and fixed to obtain a more neat and beautiful appearance.

In consideration of long-term use of the sleeping mask will cause wear and tear, especially on the parts around the eyes, eye pads 13 are provided on the inner side of the mask body 1 at a position corresponding to the eyes. The eye pads 13 are annular gaskets arranged around the outline of the eye, and the eye pads 13 are detachably installed on the inner side of the mask body 1. So, on the one hand, suitable eye pads 13 can be selected according to different users' face shapes or needs; on the other hand, if the eye pads 13 are damaged, the eye pads 13 can be replaced without affecting the normal operation of other components. In order to facilitate the installation and fixation of the eye pads 13, mounting grooves are provided on the mask body 1 where the eye pads 13 are installed, and the eye pads 13 are detachably fixed in the mounting grooves through a magnetic attraction structure.

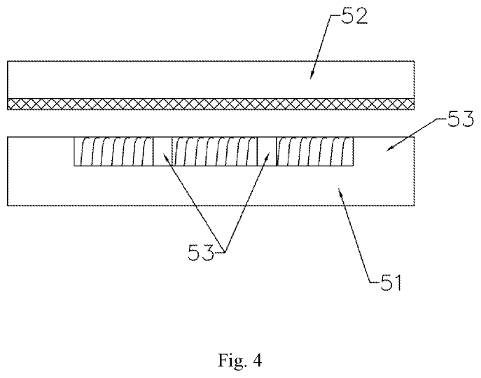

The sleep mask can be fixed on the head through a variety of structures, but the present invention is preferably implemented by using a headband with Velcro tape. Specifically, the fixing pieces 2 include headbands 5 extending from both sides of the mask body 1. The headbands 5 are connected through Velcro tape. Velcro tape is an common accessory used for repeated fixed connections on clothing, but there are some problems in the use of existing Velcro tape. For example, the hook sticking tape 51 of Velcro tape is equipped with many small hooks. In order to increase the stickiness with the loop sticking tape 52, these small hooks have strong hooking properties, even if they touch other fabrics, it will be hooked with the fabrics by the hook sticking tape 51 and when they are pulled off, it will cause hair loss on the surface of the fabrics. In order to solve the above problems, the inventor has improved the structure of the existing Velcro tape. The details are as follows, the Velcro tape is composed of a hook sticking tape 51 and a loop sticking tape 52. The hook sticking tape 51 is provided with a barrier layer 53; the barrier layer 53 is configured to be compressible. When under the non-compressed state, the barrier layer 53 prevents the hook sticking tape 51 from hooking up with the loop sticking tape 52; when under the compressed state, the hook sticking tape 51 can hook up with the loop sticking tape 52, refer to FIG. 4. Because the barrier layer 53 can isolate the small hooks of the hook sticking tape 51 from the outside, when the cloth accidentally touches the hook sticking tape 51 of the Velcro tape, the barrier layer 53 can prevent the hook from being hooked up by the small hook of the hook sticking tape 51 and sticks to the surface of the fabric. When the Velcro tape needs to be stick, the loop sticking tape 52 is placed on the hook sticking tape 51, and then the sticking part of the loop sticking tape 52 and the hook sticking tape 51 is squeezed, then the barrier layer 53 is in a compressed state, the small hook of the hook sticking tape 51 can extend out of the barrier layer 53 and hook up with the loops of the loop sticking tape 52 to achieve adhesion, and the adhesive force can keep the barrier layer 53 in a compressed state. Basing on the above design, while ensuring the sticking function of the Velcro, it can ensure the sticking function of the Velcro tape and also avoid the hair removal problem caused by other fabrics mistake touching the hook sticking tape 51. More specifically, the height of the barrier layer 53 must be equal to or higher than the height of the hooks in the hook sticking tape 51. In order to provide the barrier layer 53 with a decorative or identification function, the barrier layer 53 is distributed in the hook sticking tape 51 according to a set pattern. For example, the barrier layer 53 can be set into a decorative pattern, a trademark, etc.

In addition, this sleep mask can also be added with a brain wave recognition circuit according to the usage. By setting a brain wave sensor on the surface of the sleep mask, the activity information of the human brain is collected, and then the control circuit 3 identifies and controls the audio player 4 and/or lighting device 6 to provide further assistance. Sleep or wake functions, etc.

The above are only preferred embodiments of the present invention and do not limit the patent scope of the present invention. Any modifications, equivalent substitutions and improvements made within the concept of the present invention shall be included in the patent protection scope of the present invention.

1. A sleep mask, wherein comprising:
a mask body for covering the eyes;
fixing pieces for fixing the mask body on the head;
the mask body at least extends from the eyes to the ears; a control circuit is provided inside the mask body and includes at least an audio control module;
the audio control module includes an audio player, the audio player is set at the positions corresponding to the ears;
the mask body is composed of a front mask body and a rear mask body; the front mask body and the rear mask body are made of a sponge pad; and fixing slots for installing the control circuit and the audio player are formed in the front mask body and/or the rear mask body;
the slot for installing the control circuit extends to the edge of the mask body and forms an opening, the control circuit is inserted into the mask body from the opening and fixed.

2. The sleep mask of claim 1, wherein the outer sides of the front mask body and the rear mask body are respectively covered with fabric layers and the edges of the fabric layers cover the edges of the sponge pad of the front mask body and the rear mask body, and then glue up the front mask body and the rear mask body.

3. The sleep mask of claim 1, wherein a lighting device for guidance and/or waking up is provided on the inner side of the mask body at a position corresponding to the eyes.

4. The sleep mask of claim 3, wherein a window for installing the lighting device is provided on the inner side of the mask body facing to the eyes.

5. The sleep mask of claim 1, wherein eye pads are provided on the inner side of the mask body at a position corresponding to the eyes; the eye pads are annular gaskets arranged around the outline of the eyes; the eye pads are detachably installed on the inner side of the mask body.

6. The sleep mask of claim 1, wherein the fixing pieces include headbands extending from both sides of the mask body, and the headbands are connected by Velcro tape; the Velcro tape comprises of a hook sticking tape and a loop sticking tape, and a barrier layer is set on the hook sticking tape and the is configured to be compressible, when under the non-compressed state, the barrier layer prevents the hook sticking tape from hooking up with the loop sticking tape; when under the compressed state, the hook sticking tape can hook up with the loop sticking tape.

7. The sleep mask of claim 6, wherein the height of the barrier layer is equal to or higher than the height of the hooks in the hook sticking tape.

8. The sleep mask of claim 7, wherein the barrier layer is distributed in the hook sticking tape according to a set pattern.

9. The sleep mask of claim 7, wherein the audio control module is also connected to at least a sensor for monitoring whether the volume fed into the ears by different audio players is balanced, the sensor transmits the unbalanced volume signal fed into the ear to the audio control module, the audio control module processes the unbalanced volume signal and the audio players are adjusted to make the ears receive a balanced volume signal.
